(19) United States
(12) Patent Application Publication
(10) Pub. No.: US 2013/0006150 A1
(43) Pub. Date: Jan. 3, 2013

(54) BRUXISM DETECTION DEVICE AND BRUXISM DETECTION METHOD

(75) Inventors: Masanao SUZUKI, Yokohama (JP); Masakiyo TANAKA, Kawasaki (JP); Yasuji Ota, Yokohama (JP)

(73) Assignee: FUJITSU LIMITED, Kawasaki-shi (JP)

(21) Appl. No.: 13/613,258

(22) Filed: Sep. 13, 2012

Related U.S. Application Data
(63) Continuation of application No. PCT/JP2010/054872, filed on Mar. 19, 2010.

(51) Int. Cl. A61B 7/00

(52) U.S. Cl. 600/586

(57) ABSTRACT

A bruxism detection device includes: a sound collection unit that collects a sound produced from a subject and outputs a sound signal corresponding to the collected sound; a bruxism candidate detection unit that detects a period of a sound having a feature that is characteristic of bruxism, from the sound signal, as a bruxism candidate period; a breath detection unit that detects a period of a sound having a feature corresponding to a predetermined breathing state, from the sound signal, as a specific breathing period; and a determining unit that determines that the subject has bruxed, when the specific breathing period is present before or after the bruxism candidate period.

### CROSS-REFERENCE TO RELATED APPLICATION

This application is a continuation application and is based upon PCT/JP2010/054872, filed on Mar. 19, 2010, the entire contents of which are incorporated herein by reference.

### FIELD

The embodiments discussed herein are related to a bruxism detection device and a bruxism detection method that detects the bruxism of a subject.

### BACKGROUND

In recent years, the impact of bruxism upon health has been drawing attention. In particular, when quashing of teeth during sleep becomes longer, this might result in negative effects such as increased load upon the teeth and jaw joints, and shallow sleep, which then might trigger sleepiness during the daytime. Therefore, there is a demand for a technique for detecting bruxism during sleep.

Some techniques have been proposed to detect bruxism during sleep. For example, a technique of finding the differences in time between sounds collected by two microphones placed on two sides of the head part of a sleeping subject to determine the directions from which the sounds occur, and detecting the sound of bruxism, in addition to the snoring sound, based on the crosscorrelation coefficients of these sounds (see, for example, Japanese Laid-Open Patent Publication No. H7-184948).

In addition, a technique of identifying the sleeping conditions such as breathing, body movement, breathing during sleep, snoring, and bruxism, from comparison of an acceleration signal which has measured the acceleration of the subject's forehead part and a sound signal which has measured the sound of the forehead part, with patterns of sleeping conditions that are stored in advance (see, for example, Japanese Laid-Open Patent Publication No. 2004-187961).

### SUMMARY

However, with this technique of detecting the snoring sound and bruxism sound from differences in time and crosscorrelation coefficients between sounds collected by two microphones, features of sounds produced by the subject are not analyzed, and therefore that it is not possible to distinguish between the snoring sound and the bruxism sound. In addition, with the technique of using an acceleration signal having measured the acceleration of the subject's forehead part and a sound signal having measured the sound of the forehead part, it is necessary to set an accelerometer on the subject, and therefore the physical load upon the subject increases.

According to one embodiment, a bruxism detection device is provided. The bruxism detection device includes: a sound collection unit that collects a sound produced from a subject and outputs a sound signal corresponding to the collected sound; a bruxism candidate detection unit that detects a period of a sound having a feature that is characteristic of bruxism, from the sound signal, as a bruxism candidate period; a breath detection unit that detects a period of a sound having a feature corresponding to a predetermined breathing state, from the sound signal, as a specific breathing period; and a determining unit that determines that the subject has bruxed, when the specific breathing period is present before or after the bruxism candidate period.

According to another embodiment, a bruxism detection method is provided. The bruxism detection method includes: collecting a sound produced from a subject, and, from a sound signal corresponding to the collected sound, detecting a period of a sound having a feature that is characteristic of bruxism, as a bruxism candidate period; detecting a period of a sound having a feature that corresponds to a predetermined breathing state, from the sound signal, as a specific breathing period; and determining that the subject has bruxed, when the specific breathing period is present before or after the bruxism candidate period.

According to yet another embodiment, a computer program to cause a computer to determine whether or not a subject has bruxed is provided. The computer program includes commands for causing a computer to execute: collecting a sound produced from a subject, and, from a sound signal corresponding to the collected sound, detecting a period of a sound having a feature that is characteristic of bruxism, as a bruxism candidate period; detecting a period of a sound having a feature that corresponds to a predetermined breathing state, from the sound signal, as a specific breathing period; and determining that the subject has bruxed, when the specific breathing period is present before or after the bruxism candidate period.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is a diagram illustrating an example of a spectrum of a sound signal including a bruxism sound.

FIG. 2 is a schematic configuration diagram of a bruxism detection device according to one embodiment.

FIG. 3 is a schematic configuration diagram of a bruxism candidate detection unit.

FIG. 4A is a diagram illustrating signal power per frequency band.

FIG. 4B is a diagram illustrating a relationship between frames that are determined to be an attack sound and a period for calculating the number of attacks.

FIG. 5 is a diagram illustrating a relationship between an attack sound duration calculation analysis window and frames that are determined to be an attack sound.

FIG. 6 is an operation flowchart of a bruxism candidate detection process.

FIG. 7 is operation flowchart of a bruxism candidate detection process.

FIG. 8 is a schematic configuration diagram of a breath detection unit.

FIG. 9 is a diagram illustrating a relationship between a breath detection period and a breathing period.

FIG. 10 is an operation flowchart of a breath detection process.

FIG. 11 is an operation flowchart of a bruxism detection process.

FIG. 12 is a configuration diagram of a computer that operates as a bruxism detection device.

### DESCRIPTION OF EMBODIMENTS

A bruxism detection device according to one embodiment will be described below with reference to the accompanying drawings. This bruxism detection device collects the sounds that are produced from a subject, and, by analyzing these sounds, detects the subject's bruxism during sleep.

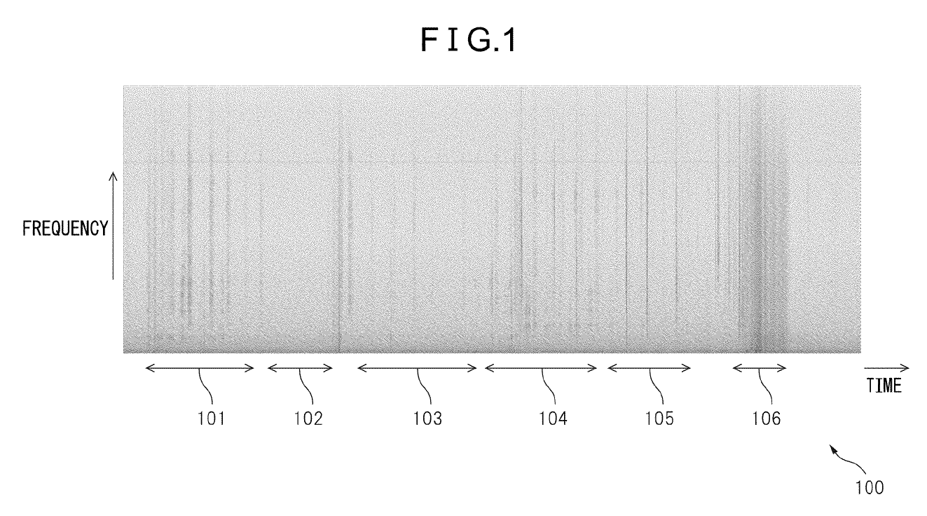

FIG. 1 is a diagram illustrating an example of a spectrum of a sound signal including a bruxism sound. In FIG. 1, the horizontal axis represents time, and the vertical axis represents frequency. Then, each line represented on the graph 100 is the spectrum signal of a sound produced from a subject while sleeping, and, when the color of a line is more dark, this means that the spectrum signal in the frequency band corresponding to the line is greater.

The periods 101 and 104 in the graph 100 contain the spectrums of the normal breathing sound. When the subject is breathing normally, the subject breathes in and breathes out in a comparatively regular cycle, so that, in these periods, spectrums are observed in a comparatively regular cycle.

On the other hand, the periods 102 and 103 correspond to the apnea state, i.e., the state in which the subject is not breathing. During the apnea state, the subject is not making the breathing sound, and therefore spectrum signals of great magnitude are not observed in the periods 102 and 103.

Furthermore, the period 105 corresponds to the state in which the subject is bruxing. Consequently, in the period 105, comparatively strong spectrums are produced continuously in short time.

Also, the period 106 corresponds to the state in which the subject rolls over. Consequently, in the period 106, comparatively strong spectrums are observed continuously.

As illustrated in FIG. 1, it is known that, when the subject bruxes, specific breathing states such as an apnea state tend to occur before and after the bruxism. In addition, the sound of bruxism has different features from the breathing sound. So, this bruxism detection device determines whether or not a subject is bruxing by analyzing the sounds produced from the subject over a predetermined period and detecting sounds having features that are characteristic of bruxism, and detecting the breathing sound to correspond to a specific breathing state before or after the sounds having features that are characteristic of bruxism.

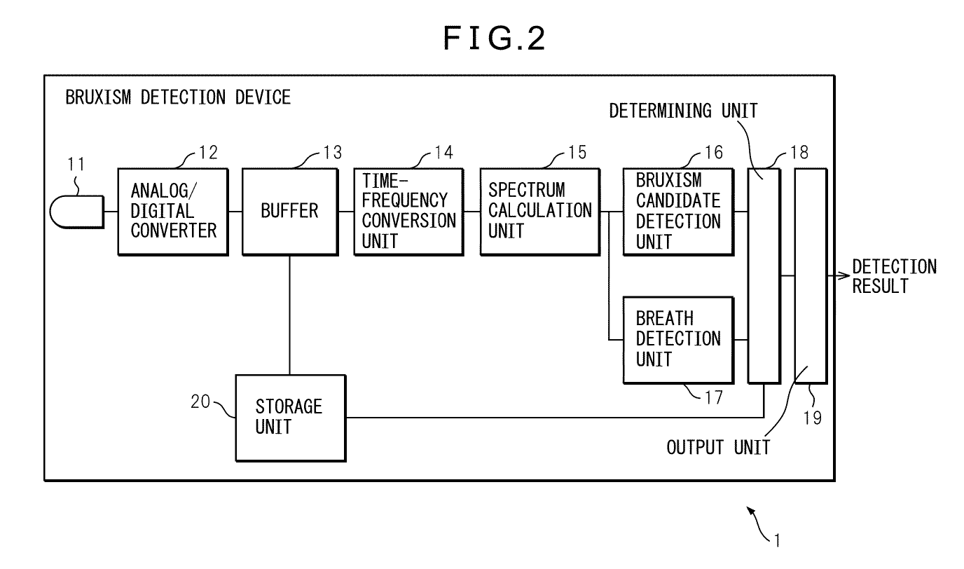

FIG. 2 is a schematic configuration diagram of a bruxism detection device according to one embodiment. The bruxism detection device 1 includes a microphone 11, an analog/digital converter 12, a buffer 13, a time-frequency conversion unit 14, a spectrum calculation unit 15, a bruxism candidate detection unit 16, a breath detection unit 17, a determining unit 18, an output unit 19, and a storage unit 20.

The microphone 11 is placed, for example, near the head part of a subject, and collects sounds that are produced around the microphone 11, including the breathing sound, bruxism sound and so on produced from the subject. Then, the microphone 11 converts the collected sounds into a sound signal, which is an electrical signal, and outputs this sound signal to the analog/digital converter 12.

The analog/digital converter 12 has, for example, an amplifying circuit and an analog/digital conversion circuit. Then, after having amplified the sound signal received from the microphone 11, the analog/digital converter 12 converts the amplified sound signal into a digital signal. The analog/digital converter 12 outputs the digitized sound signal to the buffer 13.

The buffer 13 is, for example, a readable/writable semiconductor memory. Then, the buffer 13 stores the digitized sound signal received from the analog/digital converter 12 on a temporary basis.

The time-frequency conversion unit 14, spectrum calculation unit 15, bruxism candidate detection unit 16, breath detection unit 17 and determining unit 18 are formed as separate circuits. Alternately, the time-frequency conversion unit 14, spectrum calculation unit 15, bruxism candidate detection unit 16, breath detection unit 17 and determining unit 18 may be mounted on the bruxism detection device 1, as one integrated circuit, in which circuits corresponding to these units are integrated. Furthermore, the time-frequency conversion unit 14, spectrum calculation unit 15, bruxism candidate detection unit 16, breath detection unit 17 and determining unit 18 may be function modules to be implemented by a computer program that is executed on a processor provided in the bruxism detection device 1.

The time-frequency conversion unit 14 reads the sound signal from the buffer 13 in predetermined frame length units. Then, the time-frequency conversion unit 14 generates a frequency signal by performing time-frequency conversion of the sound signal in frame length units. The frame length is set to, for example, 20 milliseconds.

For the time-frequency conversion, the time-frequency conversion unit 14 uses, for example, the fast Fourier transform (FFT). The frequency signal Xₙ(k) acquired with respect to the n-th frame can be represented, for example, by the following equation:

Xₙ(k)=Rₙ(k)+j·Iₙ(k) (k=0, 1, . . . , K−1) (1)

Rₙ(k) represents the frequency signal of the real part of the frequency band k, and Iₙ(k) represents the frequency signal of the imaginary part of the frequency band k. Also, K is the total number of frequency bands. In this case, frequency bands that are equal to or below the Nyquist frequency are represented by 0 to (K/2−1).

Note that, for the time-frequency conversion, the time-frequency conversion unit 14 may use the discrete cosine transform, modified discrete cosine transform or Wavelet transform, instead of the FFT.

The time-frequency conversion unit 14 outputs the generated frequency signals Xₙ(k) to the spectrum calculation unit 15.

From the frequency signals Xₙ(k) received from the time-frequency conversion unit 14, the spectrum calculation unit 15 generates the spectrum signal Sₙ(k) of each frequency band k, in frame length units, according to the following equation:

Sₙ(k)=|Xₙ(k)|²=Rₙ(k)²+Iₙ(k)² (k=0, 1, . . . , K−1) (2)

Note that K is the total number of frequency bands.

The spectrum calculation unit 15 outputs the generated spectrum signals Sₙ(k) to the bruxism candidate detection unit 16 and breath detection unit 17.

By detecting features that are characteristic of the sound of bruxism that is produced by the bruxism, based on the spectrum signals Sₙ(k) received from the spectrum calculation unit 15, the bruxism candidate detection unit 16 detects a signal period having the features as a bruxism candidate.

The sound of bruxism meets the following conditions.

(1) The sound of bruxism is greater than the background noise. In particular, in a specific frequency band (for example, 3 kHz to 4 kHz), the sound of bruxism is greater than the background noise.

(2) The duration of the sound of bruxism is, in general, 0.1 second to several seconds.

(3) The sound of bruxism has little periodicity.

(4) The sound of bruxism is an attack-like sound that occurs continuously in short time.

Therefore, the bruxism sound candidate detection unit 16 determines whether or not all of these conditions (1) to (4) are met, from the spectrum signals. Then, the bruxism candidate detection unit 16 makes a signal period to satisfy all of these conditions (1) to (4) a bruxism candidate.

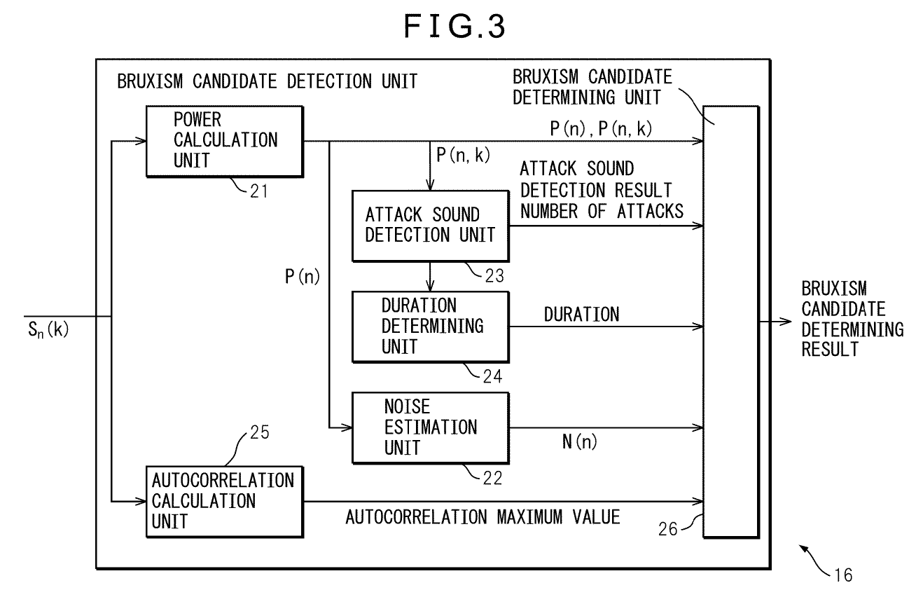

FIG. 3 is a schematic configuration diagram of the bruxism candidate detection unit 16. The bruxism candidate detection unit 16 includes a power calculation unit 21, a noise estimation unit 22, an attack sound detection unit 23, a duration determining unit 24, an autocorrelation calculation unit 25, and a bruxism candidate determining unit 26. Amongst these, the power calculation unit 21, noise estimation unit 22, attack sound detection unit 23, duration determining unit 24 and autocorrelation calculation unit 25 are each an example of a feature extraction unit that extracts a feature related to the sound of bruxism, from the spectrum signals.

The power calculation unit 21 calculates the whole-band signal power value P(n), which is an indicator to represent the volume of sound in the present frame, from the spectrum signal Sₙ(k) of the present frame, according to the following equation:

$$P ( n ) = {\sum }_{k = 0}^{K / 2 - 1} P ( n , k ) = {\sum }_{k = 0}^{K / 2 - 1} {{S}_{n} ( k )}^{2} \qquad ( 3 )$$

Note that, n is the frame number to correspond to the present frame, and P(n,k) is the power of the frequency band k in the present frame. K is the total number of frequency bands.

The power calculation unit 21 outputs the whole-band signal power value P(n), to the noise estimation unit 22 and bruxism candidate determining unit 26. The power calculation unit 21 also outputs the signal power P(n, k) of each frequency band, to the attack sound detection unit 23 and bruxism candidate determining unit 26.

The noise estimation unit 22 calculates the background noise power value, which corresponds to the background noise contained in the present frame. While the subject is sleeping, the subject is estimated to be in a comparatively quiet location. Consequently, it is estimated that the background noise that is produced in the surrounding environment is smaller than the sounds produced by the subject, and the fluctuation of the background noise power is small.

Therefore, when the whole-band signal power value of the present frame is substantially equal to a past background noise power value, the noise estimation unit 22 estimates that the signal power value is the background noise. Then, the noise estimation unit 22 finds an average of the past background noise power value and the whole-band signal power value of the present frame, and finds the background noise power value with respect to the present frame. On the other hand, when the whole-band signal power value of the present frame is greater than a past background noise power value, the noise estimation unit 22 estimates that the whole-band signal power value includes sounds other than the background noise, for example, the breathing sound or bruxism sound produced by the subject. Then, the noise estimation unit 22 makes the past background noise power value the background noise power value of the present frame.

For example, the background noise power of the previous frame is represented by N(n−1). In this case, the noise estimation unit 22 calculates the background noise power N(n) of the present frame according to the following equations:

N(n)=α·N(n−1)+(1−α)·P(n)P(n)<N(n−1)×γN(n)=N(n−1)P(n)≧N(n−1)×γ (4)

Note that, α is a forgetting factor and is set to, for example, α=0.9, γ is a constant and is set to, for example, 1.5 to 2.0.

The noise estimation unit 22 outputs the background noise power N(n) of the present frame to the bruxism candidate determining unit 26, and stores the background noise power N(n) of the present frame, until the background noise power of the next frame is calculated.

The attack sound detection unit 23 finds the difference between signal power value of the present frame and the signal power value of the previous frame, per frequency band, thereby determining whether or not the present frame corresponds to an attack sound.

The attack sound tends to be laud instantaneously over a wide frequency band. Therefore, the attack sound detection unit 23 calculates the signal power difference between the present frame n and the previous frame (n−1), per frequency band, according to the following equation:

ΔP(k)=P(n,k)−P(n−1,k) (k=0, 1, . . . , K/2−1) (5)

Note that K is the total number of frequency bands. P(n, k) and P(n−1, k) are the signal power value of the frequency band k in the present frame n, and the signal power value of the frequency band k in the previous frame (n−1), respectively. Then, ΔP(k) is the signal power difference in the frequency band k.

The attack sound detection unit 23 determines, for each frequency band, whether or not the acquired signal power difference ΔP(k) is equal to or greater than a predetermined power threshold value. Then, the attack sound detection unit 23 finds the number of frequency bands where the signal power difference ΔP(k) is equal to or greater than the predetermined power threshold value, as the number of bands with increased power. When the number of bands with increased power is equal to or greater than a predetermined threshold value for the number of bands, the attack sound detection unit 23 determines that the present frame includes an attack sound. Then, the attack sound detection unit 23 stores the frame number that is determined to be an attack sound for certain period of time. This certain period is set to, for example, the period to count the number of frames determined to be an attack sound, as will be described later.

Note that the power threshold value is set to, for example, a value to match 3 to 6 dB. Assuming that one frame of a sound signal is represented by 256 sample points, the threshold value for the number of bands is set to, for example, 100, when a frequency signal is found by the FFT. Alternately, the threshold value for the number of bands may be set as a ratio to the whole band spectrum of the sound collected by the microphone 11. In this case, for example, when the Nyquist frequency is Fs, the threshold value for the number of bands is set to, for example, a value to match 0.8 Fs.

Next, the attack sound detection unit 23 calculates the number of frames that are determined to be an attack sound and are included in a period of a predetermined unit time length whose end is the present frame, as the number of attacks in the present frame. This unit time is set to, for example, 1 second.

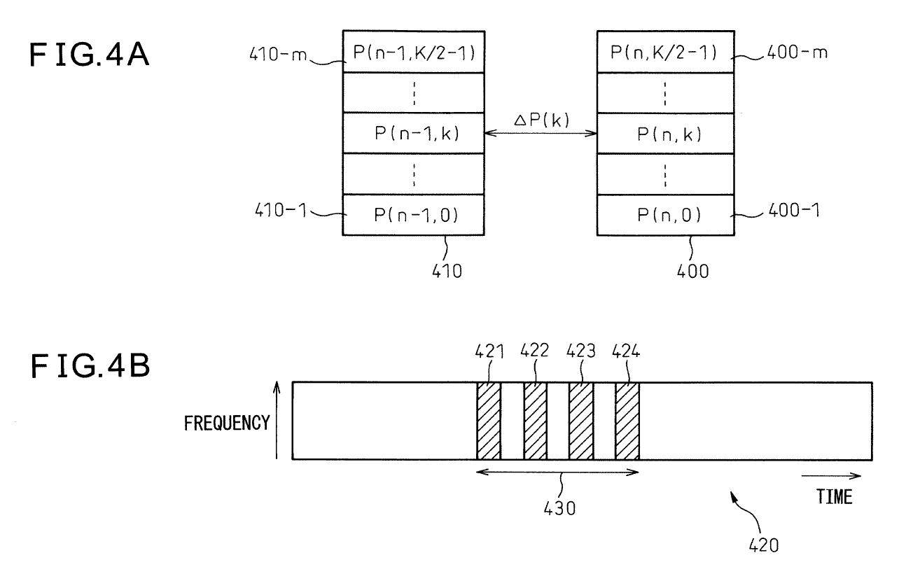

FIG. 4A is a diagram illustrating the signal power per frequency band. FIG. 4B is a diagram illustrating the relationship between frames that are determined to be an attack sound and the period for calculating the number of attacks.

In FIG. 4 A, frame 400 is the present frame, and frame 410 is the previous frame. Blocks 400-1 to 400-m included in the frame 400 represent the signal power of each frequency band included in the frame 400. Similarly, blocks 410-1 to 410-m included in the frame 410 represent the signal power of each frequency band included in the frame 410. The attack sound detection unit 23 calculates the signal power difference ΔP(k) for each frequency band that is the same between the frame 400 and the frame 410.

In FIG. 4B, the graph 420 represents the spectrums of sounds collected by the microphone 11. The horizontal axis represents time, and the vertical axis represents frequency. The blocks 421 to 424 depicted by hatching are frames that are determined to be an attack sound. Then, frame corresponding to the block 424 is the present frame. Period 430 for counting the number of attacks is set such that the present frame comes at the end of the period 430. In this case, the period 430 includes four frames to be determined to be an attack sound.

The attack sound detection unit 23 reports the result of determining whether or not the present frame corresponds to an attack sound, to the duration determining unit 24. In addition, the attack sound detection unit 23 output the number of attacks to the bruxism candidate determining unit 26.

The duration determining unit 24 finds the length of the period in which an attack sound occurs repeatedly. When the present frame is determined to be an attack sound, the duration determining unit 24 sets up an analysis window of which the present frame comes at the end. This analysis window is set longer than the length of a frame, which is the unit for executing the time-frequency conversion by the time-frequency conversion unit 14, and is, preferably, set longer than the maximum value of the period when the sound of bruxism generally lasts. For example, the analysis window is set to, for example, 10 seconds.

The duration determining unit 24 determines whether or not the analysis window includes a frame that is determined to be an attack sound. Then, when at least one frame that is determined to be an attack sound is included, the duration determining unit 24 shifts the analysis window to previous time by time ΔT. Then, the duration determining unit 24 sets the attack sound duration T to ΔT. Note that ΔT is set to, for example, a value to be shorter than the minimum value of the period when the sound of bruxism lasts, for example, 40 milliseconds.

The duration determining unit 24 determines whether or not a frame that is determined to be an attack sound are included in the analysis window shifted to previous time by ΔT. Then, when at least one frame that is determined to be an attack sound is included in the analysis window, the duration determining unit 24 adds ΔT to the duration T, and shifts the analysis window to previous time by time ΔT again.

The duration determining unit 24 repeats the same process until no frame that is determined to be an attack sound is included in the analysis window. Then, when a frame that is determined to be an attack sound is no longer included in the analysis window, the duration determining unit 24 determines the duration T at that point in time as the duration of attack sounds in the present frame.

On the other hand, when the present frame is determined not to be an attack sound, the duration determining unit 24 makes the value, that is given by subtracting the frame length from the duration calculated in the previous frame, the duration of attack sounds in the present frame. However, when the duration calculated in this way assumes a negative value, the duration determining unit 24 sets the duration of attack sounds in the present frame to 0.

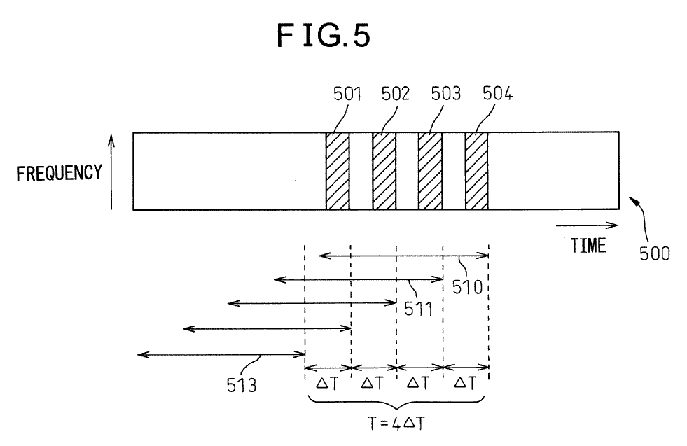

FIG. 5 is a diagram illustrating the relationship between the attack sound duration calculation analysis window and frames that are determined to be an attack sound. In FIG. 5, graph 500 represents the spectrums of sounds collected by the microphone 11. The horizontal axis represents time, and the vertical axis represents frequency. Blocks 501 to 504 depicted by hatching are frames that have been determined that the frames correspond to an attack sound. Then, the frame corresponding to the block 504 is the present frame. Analysis window 510 for determining the duration of attack sounds is first set such that the present frame 504 comes at the end of the analysis window. In this case, four frames that are determined to be an attack sound are included in the analysis window 510. Therefore, the duration determining unit 24 sets a new analysis window 511 at the time when the analysis window 510 is shifted to previous time by ΔT. The analysis window 511 also includes frames that are attack sounds. Therefore, the duration determining unit 24 sets a new analysis window 512 at the time when the analysis window 511 is shifted to previous time by ΔT. By shifting the analysis window in this way, the analysis window 513, which is shifted by 4ΔT from the analysis window 510, no longer includes a frame that is an attack sound. Therefore, the duration determining unit 24 sets duration T of attack sounds to 4ΔT.

The duration determining unit 24 stores the duration of attack sounds determined with respect to the present frame on a temporary basis, and outputs the duration of attack sounds found with respect to the present frame to the bruxism candidate determining unit 26. In addition, to check the duration of attack sounds in the next frame and onward, the duration determining unit 24 stores the result of determining whether or not the present frame is an attack sound, in association with the number of the present frame.

The autocorrelation calculation unit 25 calculates the autocorrelation coefficient acor(d) between the spectrum signal Sₙ(k) of the present frame n and the spectrum signal S_{n-d}(k) of a past frame (n−d), according to the following equation, as an indicator of the periodicity of sounds collected by the microphone 11:

$$acor ( d ) = \frac{{\sum }_{k = 0}^{K / 2 - 1} {S}_{n} ( k ) {S}_{n - d} ( k )}{{\sum }_{k = 0}^{K / 2 - 1} {{S}_{n} ( k )}^{2}} \qquad ( 6 )$$

d is the variable to represent delay, given in frame units. For example, when d=1, S_{n-d}(k) is the previous frame of the present frame n. Also, k is the frequency band, and K is the total number of frequency bands.

The autocorrelation calculation unit 25 calculates the autocorrelation coefficient by changing d in the range of 1 to dₘₐₓ. Then, the autocorrelation calculation unit 25 finds the maximum value of the autocorrelation coefficient, and outputs that maximum value to the bruxism candidate determining unit 26. Note that dₘₐₓ is set to, for example, a number of frames corresponding to 0.1 second to several seconds, which is the period in which the sound of bruxism lasts.

When a value related to a feature of the bruxism sound, calculated by each unit of the bruxism candidate detection unit 16, fulfills a predetermined condition, the bruxism candidate determining unit 26 determines that the signal period to include the present frame is a bruxism candidate. With the present embodiment, values related to the features of the bruxism sound include the whole-band signal power, the background noise power, the signal power of a specific frequency band, the duration of attack sounds, the autocorrelation coefficient maximum value and the number of attacks. Then, when all of the conditions (1) to (4) as described above are met, from these values, the bruxism candidate determining unit 26 determines that the signal period to include the present frame is a bruxism candidate. On the other hand, when at least one of the conditions (1) to (4) is not met, the bruxism candidate determining unit 26 determines that the signal period to include the present frame is not a bruxism candidate. Note that this signal period can be made, for example, a period to include only the present frame. Alternately, this signal period can be used as a signal period corresponding to the duration of attack sounds with respect to the present frame. In the following example, the signal period determined to be bruxism candidate includes only the present frame.

For example, for the condition (1), the bruxism candidate determining unit 26 determines whether or not the whole-band signal power value is greater than the background noise power value. Further, the bruxism candidate determining unit 26 determines whether or not the signal power value of a specific frequency band is greater than a predetermined threshold value Th1. When the whole-band signal power value is greater than the background noise power value and the signal power value of a specific frequency band is equal to or greater than the predetermined threshold value Th1, the bruxism candidate determining unit 26 determines that the condition related to the sound volume of the bruxism sound is fulfilled. Note that the specific frequency band is set, for example, in a range between 3 kHz and 4 kHz. Also, in the bruxism sound, the sound of a specific frequency band is greater than the sounds of the other frequency bands, so that the threshold value Th1 is set, for example, to the average power of the whole frequency band or the background noise power. Alternately, the threshold value Th1 may be the value given by adding a predetermined bias (for example, 3 dB or greater) to the average power of the whole frequency band or the background noise power.

For the condition (2), when the duration of attack sounds is equal to or longer than a threshold value Th2, the bruxism candidate determining unit 26 determines that the condition related to the duration of the bruxism sound is fulfilled. As described above, the sound of bruxism tends to last 0.1 second to several seconds. Therefore, the threshold value Th2 is set to a number of frames corresponding to 0.1 second to several seconds.

For the condition (3), when the maximum value of the autocorrelation coefficient is equal to or lower than a threshold value Th3, the bruxism candidate determining unit 26 determines that the condition related to the periodicity of the bruxism sound is fulfilled. When the periodicity is lower, the maximum value of the autocorrelation coefficient also decreases. So, the threshold value Th3 is set to, for example, 0.5.

For the condition (4), when the number of attacks is equal to or greater than a threshold value Th4, the bruxism candidate determining unit 26 determines that the condition related to the continuity of the bruxism sound is fulfilled. For example, the threshold value Th4 is set to the minimum number of attack sounds that occur per unit time during the bruxism. For example, the threshold value Th4 is set to, for example, an integer equal to or greater than 2—for example, 3.

The bruxism candidate determining unit 26 outputs the result of determining whether or not the signal period including the present frame is a bruxism candidate, to the determining unit 18, with the present frame number.

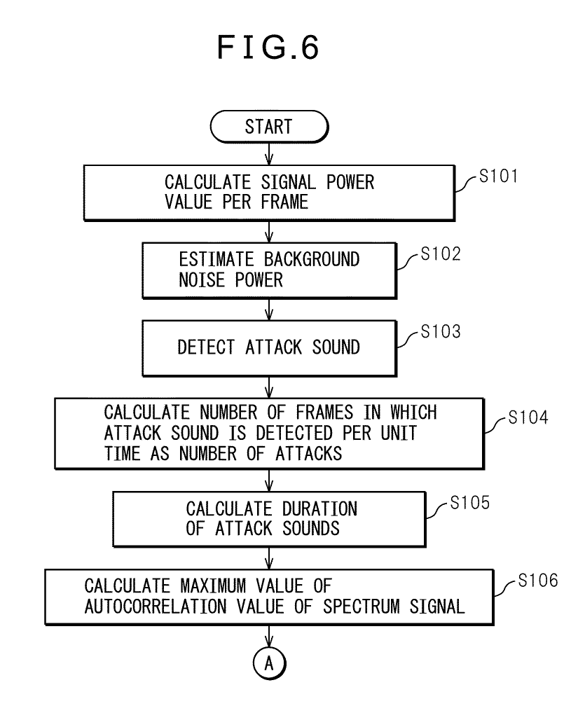

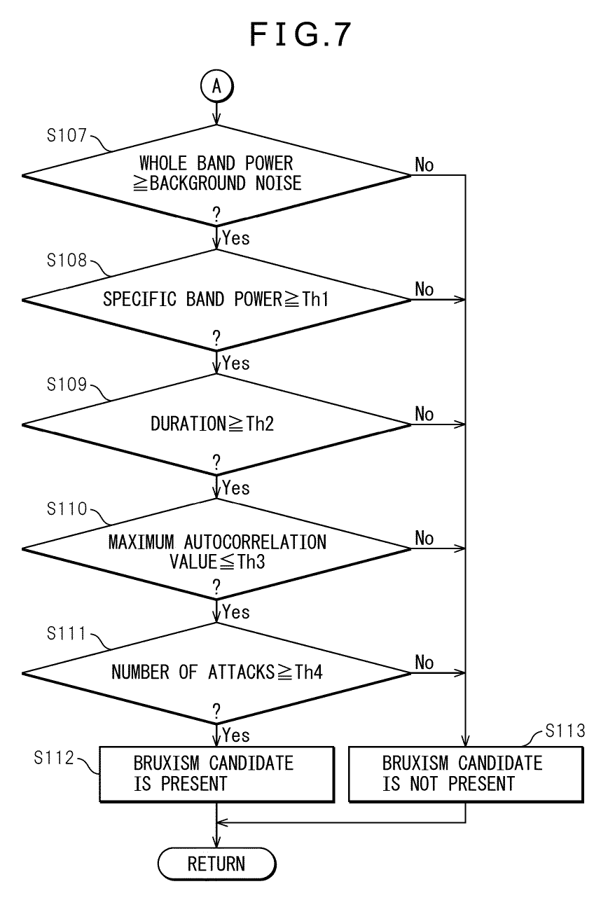

FIG. 6 and FIG. 7 are operation flowcharts of the bruxism candidate detection process. Note that the bruxism candidate detection unit 16 executes the bruxism candidate detection process on a per frame.

As illustrated in FIG. 6, the power calculation unit 21 calculates the whole-band signal power value, and the signal power value of each frequency band, of the present frame (step S101). Then, the power calculation unit 21 outputs the whole-band signal power value to the noise estimation unit 22 and the bruxism candidate determining unit 26. In addition, the power calculation unit 21 outputs the signal power value of each frequency band to the attack sound detection unit 23 and the bruxism candidate determining unit 26.

Upon receiving the whole-band signal power value of the present frame, the noise estimation unit 22 estimates the background noise power with respect to the present frame, based on the whole-band signal power value and the whole-band signal power value of a past frame (step S102). Then, the noise estimation unit 22 stores the background noise power of the present frame on a temporary basis, and outputs the background noise power of the present frame to the bruxism candidate determining unit 26.

In addition, the attack sound detection unit 23 detects an attack sound based on the difference between the signal power value of each frequency band in the present frame, and the signal power value of a corresponding frequency band in a past frame (step S103). Further, the attack sound detection unit 23 stores the signal power value of each frequency band of the present frame, on a temporary basis, to use in the attack sound detection in the next frame.

Furthermore, the attack sound detection unit 23 calculates the number of frames where an attack sound is detected per unit time, as the number of attacks (step S104). Then, the attack sound detection unit 23 outputs the result of determining whether or not the present frame correspond to an attack sound, to the duration determining unit 24 and the bruxism candidate determining unit 26, with the number of the present frame. Further, the attack sound detection unit 23 outputs the number of attacks to the bruxism candidate determining unit 26.

The duration determining unit 24 calculates the duration of attack sounds (step S105). Then, the duration determining unit 24 outputs the duration to the bruxism candidate determining unit 26.

The autocorrelation calculation unit 25 calculates the maximum value of the autocorrelation values between the spectrum signal of the present frame and the spectrum signal of a past frame, as an indicator for representing the periodicity of the sound signal (step S106). Then, the autocorrelation calculation unit 25 outputs the maximum value of the autocorrelation values, to the bruxism candidate determining unit 26. Further, the autocorrelation calculation unit 25 stores the spectrum signal of the present frame, on a temporary basis, to utilize in the autocorrelation value calculation in the next frame.

As illustrated in FIG. 7, the bruxism candidate determining unit 26 determines whether or not the whole band power is equal to or greater than the background noise (step S107). When the whole band power is lower than the background noise (step S107—No), the bruxism candidate determining unit 26 determines that the present frame is not a bruxism candidate (step S113).

On the other hand, when the whole band power is equal to or greater than the background noise (step S107—Yes), the bruxism candidate determining unit 26 determines whether or not the power of a specific band is equal to or greater than a threshold value Th1 (step S108). When the power of a specific band is lower than the threshold value Th1 (step S108—No), the bruxism candidate determining unit 26 determines that the present frame is not a bruxism candidate (step S113).

On the other hand, when the power of a specific band is equal to or greater than the threshold value Th1 (step S108—Yes), the bruxism candidate determining unit 26 determines whether or not the duration of attack sounds is equal to or longer than the threshold value Th2 (step S109). When the duration of attack sounds is shorter than the threshold value Th2 (step S109—No), the bruxism candidate determining unit 26 determines that the present frame is not a bruxism candidate (step S113).

On the other hand, when the duration of attack sounds is equal to or longer than the threshold value Th2 (step S109—Yes), the bruxism candidate determining unit 26 determines whether or not the maximum autocorrelation value, which is an indicator of periodicity, is equal to or greater than a threshold value Th3 (step S110). When the maximum autocorrelation value is greater than the threshold value Th3 (step S110—No), the bruxism candidate determining unit 26 determines that the present frame is not a bruxism candidate (step S113).

On the other hand, when the maximum autocorrelation value is equal to or lower than the threshold value Th3 (step S110—No), the bruxism candidate determining unit 26 determines whether or not the number of attacks is equal to or greater than a threshold value Th4 (step S111).

When the number of attacks is equal to or greater than the threshold value Th4, at the present frame, all of the conditions (1) to (4), which correspond to the sound of bruxism, are fulfilled. Therefore, the bruxism candidate determining unit 26 determines that the present frame is a bruxism candidate (step S112). Then, the bruxism candidate determining unit 26 outputs a flag indicating the presence of a bruxism candidate, to the determining unit 18, as the result of determining whether or not the present frame is a bruxism candidate, with the number of the present frame.

On the other hand, when the number of attacks is lower than the threshold value Th4, the bruxism candidate determining unit 26 determines that the present frame is not a bruxism candidate (step S113). Then, the bruxism candidate determining unit 26 outputs a flag indicating the absence of a bruxism candidate, to the determining unit 18, as the result of determining whether or not the present frame is a bruxism candidate, with the number of the present frame.

After step S112 or S113, the bruxism candidate determining unit 26 finishes the process. Note that the bruxism candidate determining unit 26 may change the order of executing the processes of step S107 to S111 in any way.

The breath detection unit 17 detects signal periods corresponding to a specific breathing state, such as an apnea state, based on spectrum signals.

The breathing sound occurs at comparatively regular intervals. In addition, when no sound is produced from the subject, i.e., when there is only background noise, the autocorrelation of the spectrum is higher than the sound when the subject is bruxing. With the present embodiment, the breath detection unit 17 detects a period in which the spectrum has high autocorrelation as a breathing period in which the subject is breathing, and, by finding the difference in time between breathing periods, detects the period of the apnea state, as a signal period corresponding to a specific breathing state.

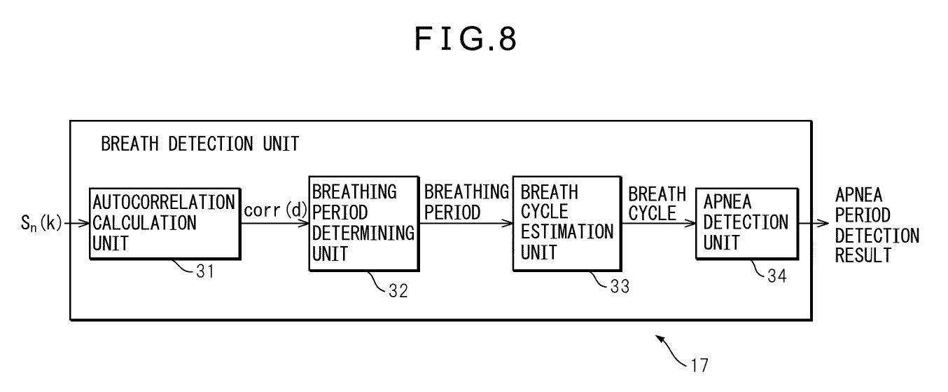

FIG. 8 is a schematic configuration diagram of the breath detection unit 17. The breath detection unit 17 includes an autocorrelation calculation unit 31, a breathing period determining unit 32, a breath cycle estimation unit 33, and an apnea detection unit 34. Then, the breath detection unit 17 acquires spectrum signal per breath detection period, from the spectrum calculation unit 15, and finds the period of the apnea state per breath detection period. Note that the breath detection period is set approximately to a period to include several breaths—for example, 10 seconds. Further, the breath detection unit 17 acquires the frame number identifying the breath detection period, from the spectrum calculation unit 15. Note that the frame number to identify the breath detection period is, for example, the number of the first or the last frame in the breath detection period.

The autocorrelation calculation unit 31 calculates the autocorrelation coefficient per frame unit, as an indicator of the periodicity of the spectrum signal in the breath detection period.

The autocorrelation calculation unit 31 sets each frame included in the breath detection period as a frame of interest, in order, from the past, for example. Then, the autocorrelation calculation unit 31 calculates the autocorrelation coefficient corr(d) between the spectrum signal Sₙ(k) of the frame of interest n, and the spectrum signal S_{n-d}(k) of a past frame (n-d), as an indicator of the periodicity of sounds collected by the microphone 11, according to the following equation:

$$corr ( d ) = \frac{{\sum }_{k = 0}^{K / 2 - 1} {S}_{n} ( k ) {S}_{n - d} ( k )}{{\sum }_{k = 0}^{K / 2 - 1} {{S}_{n} ( k )}^{2}} \qquad ( 7 )$$

d is the variable to represent delay, given in frame units. For example, given that d=1, S_{n-d}(k) is the previous frame of the frame of interest n. In addition, k is the frequency band, and K is the total number of frequency bands.

The autocorrelation calculation unit 31 calculates the autocorrelation coefficient of the frame of interest by changing d in the range between −dₘₐₓ₂ and dₘₐₓ₂. Then, the autocorrelation calculation unit 31 outputs the autocorrelation coefficient of the frame of interest with respect to the value of each d, to the breathing period determining unit 32. Note that the dₘₐₓ₂ is set to, for example, a number of frames corresponding to the breath detection period.

The breathing period determining unit 32 determines the breathing periods, which are periods in which the subject is breathing, based on the autocorrelation coefficient of each frame in the breath detection period. The sound when the subject is breathing is generally bigger than the sound when the subject is not breathing and there is only background noise. So, the breathing period determining unit 32 calculates the autocorrelation coefficient corr(d) with respect to each frame in the breath detection period. The breathing period determining unit 32 sets the frame when the autocorrelation coefficient corr(d) is the maximum, as the frame of interest. Then, the breathing period determining unit 32 detects the frame of interest, when the autocorrelation coefficient corr(d) with respect to the frame of interest is equal to or greater than a predetermined breathing sound threshold value, and detects the frame corresponding to the delay d for the frame of interest. Then, the breathing period determining unit 32 determines the periods when the detected frames continue as one breathing period.

Alternately, given the frames in the breath detection period, the breathing period determining unit 32 may detect all the frames in which the autocorrelation coefficient corr(d) is equal to or greater than the breathing sound threshold value, and determine the periods when the detected frames continue as one breathing period.

The breathing sound threshold value is set to a noise average correlation value, which is an average value of the autocorrelation values calculated with respect to the spectrums including only the background noise, and the value given by adding a predetermined bias value (for example, 0.1) to the noise average correlation value. Alternately, the breathing sound threshold value is set to a value whereby the presence of autocorrelation can be acknowledged, for example, 0.5.

The breathing period determining unit 32 outputs the frame number at the center of each breathing period, to the breath cycle estimation unit 33.

The breath cycle estimation unit 33 finds the interval between the breathing periods, i.e., the interval between the center frame of a specific breathing period, and the center frame of its previous breathing period, as the breath cycle. Note that, as for the breathing period that is detected first in the present breath detection period, the breath cycle estimation unit 33 makes the interval with the breathing period that is detected last, among the breath detection periods earlier than the present breath detection period, the breath cycle.

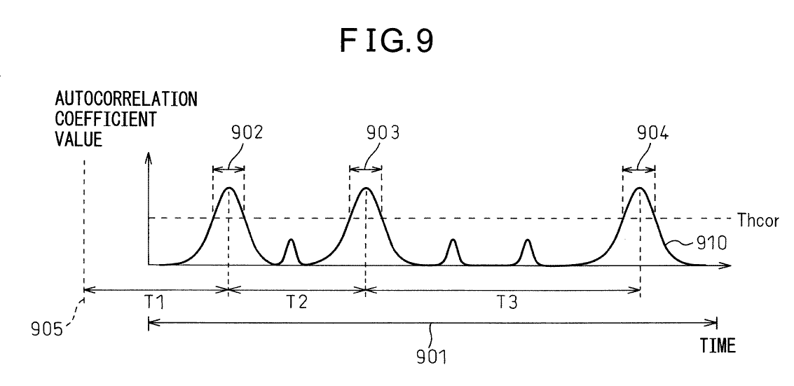

FIG. 9 is a diagram illustrating the relationship between a breath detection period and breathing periods. In FIG. 9, the horizontal axis represents time, and the vertical axis represents the autocorrelation coefficient value. The period designated by the arrow 901 represents a breath detection period. Then, the graph 910 represents the autocorrelation coefficient calculated with respect to the frame when the autocorrelation value is the maximum, among the frames in the breath detection period 901. The threshold value Thcor is the breathing sound threshold value. In this example, in the periods 902 to 904, the autocorrelation coefficient is equal to or greater than the breathing sound threshold value. Consequently, the periods 902 to 904 are each detected as a breathing period. Then, the breath cycle T2 for the breathing period 903 is the difference in time between the center of the breathing period 902 and the center of the breathing period 903. Similarly, the breath cycle T3 for the breathing period 904 is the time difference between the center of the breathing period 903 and the center of the breathing period 904. On the other hand, in the breathing period 902, there is no breathing period to precede the breathing period 902 in the breath detection period 901. So, the breath cycle T1 of the breathing period 902 is the time difference between the center of the breathing period 902 and the center 905 of the breathing period that is detected last in the previous breath detection period from the breath detection period 901.

The breath cycle estimation unit 33 outputs the breath cycle that is found with respect to each breathing period in the present breath detection period, to the apnea detection unit 34.

The apnea detection unit 34 compares each breath cycle in the present breath detection period with a predetermined apnea detection threshold value. Then, when any of the breath cycles is equal to or longer than the apnea detection threshold value, the apnea detection unit 34 determines that the breath cycle corresponds to an apnea period. Then, the apnea detection unit 34 outputs the result of determining whether or not there is an apnea period in the present breath detection period, to the determining unit 18. Note that the apnea detection threshold value is set to, for example, to a number of frames corresponding to 10 seconds.

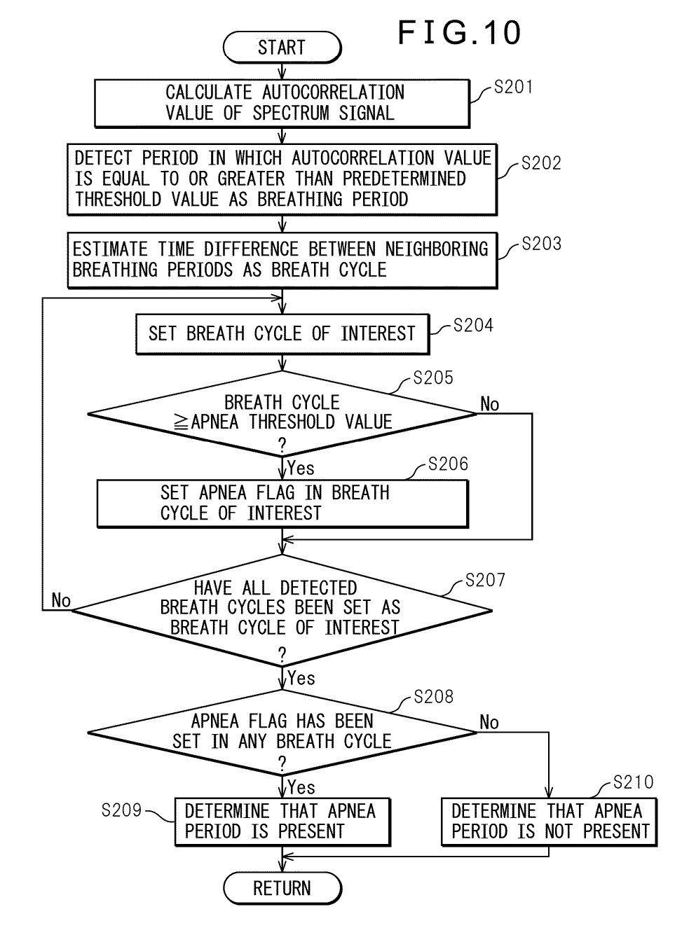

FIG. 10 is an operation flowchart of the breath detection process executed by the breath detection unit 17. Note that the breath detection unit 17 executes this breath detection process per breath detection period.

The autocorrelation calculation unit 31 calculates the autocorrelation value of the spectrum signal with respect to each frame in the breath detection period (step S201). Then, the autocorrelation calculation unit 31 outputs the autocorrelation value of each frame to the breathing period determining unit 32.

The breathing period determining unit 32 detects a period in which the autocorrelation value is equal to or greater than a breathing sound threshold value as a breathing period (step S202). The breathing period determining unit 32 outputs the frame number at the center of each breathing period to the breath cycle estimation unit 33.

For each breathing period in the present breath detection period, the breath cycle estimation unit 33 estimates the difference between the breathing period and its previous breathing period, as the breath cycle for the breathing period (step S203). The breath cycle estimation unit 33 outputs the breath cycle determined with respect to each breathing period in the present breath detection period, to the apnea detection unit 34.

The apnea detection unit 34 sets the breath cycle of interest among the breath cycles which have not been set as a breach cycle of interest (step S204). Then, the apnea detection unit 34 determines whether or not the breath cycle of interest is equal to or greater than an apnea detection threshold value (step S205). When the breath cycle of interest is equal to or longer than the apnea detection threshold value (step S205—Yes), the apnea detection unit 34 sets an apnea flag in the breath cycle of interest (step S206).

After step S206, alternately, the breath cycle that is of interest in step S205 is shorter than the apnea detection threshold value, the apnea detection unit 34 determines whether or not all the detected breath cycles have been set as a breath cycle of interest (step S207). When there is a breath cycle that has not been set as a breath cycle of interest (step S207—No), the apnea detection unit 34 repeats the processes of steps S204 to S207.

On the other hand, when all the breath cycles have been set as a breath cycle of interest (step S207—Yes), the apnea detection unit 34 determines whether or not any of the breath cycles is set to an apnea flag (step S208).

When any of the breath cycles is set to an apnea flag (step S207—Yes), the apnea detection unit 34 outputs the result of determining that an apnea period is present, to the determining unit 18, with a frame number to designate the present breath detection period (step S209). On the other hand, when none of the breath cycles is set to an apnea flag (step S207—No), the apnea detection unit 34 outputs the result of determining that there is no apnea period, to the determining unit 18, with a frame number to designate the present breath detection period (step S210). After step S209 or S210, the breath detection unit 17 finishes the breath detection process.

The determining unit 18 determines whether or not the subject is bruxing, based on the signal period determined to be a bruxism candidate and the apnea period. As described above, there is a tendency that the subject enters an apnea state before or after bruxing. Therefore, the determining unit 18 stores the results of determining whether or not there is an apnea period, with respect to the latest several breath detection periods. Further, the determining unit 18 stores the frame number corresponding to a signal period determined to be a bruxism candidate, for a certain period. Then, when an apnea period is present before or after the signal period that is determined to be a bruxism candidate, the determining unit 18 determines that the subject is bruxing. For example, when there is an apnea period in one minute either before or after a signal period that is determined to be a bruxism candidate, the determining unit 18 determines that the subject is bruxing.

Upon determining that the subject is bruxing, the determining unit 18 outputs a bruxism detection signal representing the determined result, to the output unit 19. In addition, from the sound signals stored in the buffer 13, the determining unit 18 may read a bruxism candidate signal period as of when bruxism is detected, and sound signals of a predetermined period before and after the bruxism candidate signal period, from the buffer 13, and store them in the storage unit 20.

The output unit 19 includes an interface circuit for connecting the bruxism detection device 1 with other devices. Then, the output unit 19 outputs a bruxism detection signal received from the determining unit 18, to other devices. Furthermore, the output unit 19 may read the sound signal of the frame when bruxism is detected, from the storage unit 20, and outputs that signal to other devices.

The storage unit 20 may have at least one of, for example, a semiconductor memory, a magnetic disk device and an optical disk device. Then, the storage unit 20 stores the result of determining whether or not bruxism is detected, received from the determining unit 18. The storage unit 20 may store the sound signals of the frame when bruxism is detected, and frames before and after the frame when bruxism is detected.

Furthermore, the storage unit 20 may store, on a temporary basis, various data to be calculated by the time-frequency conversion unit 14, spectrum calculation unit 15, bruxism candidate detection unit 16 and breath detection unit 17.

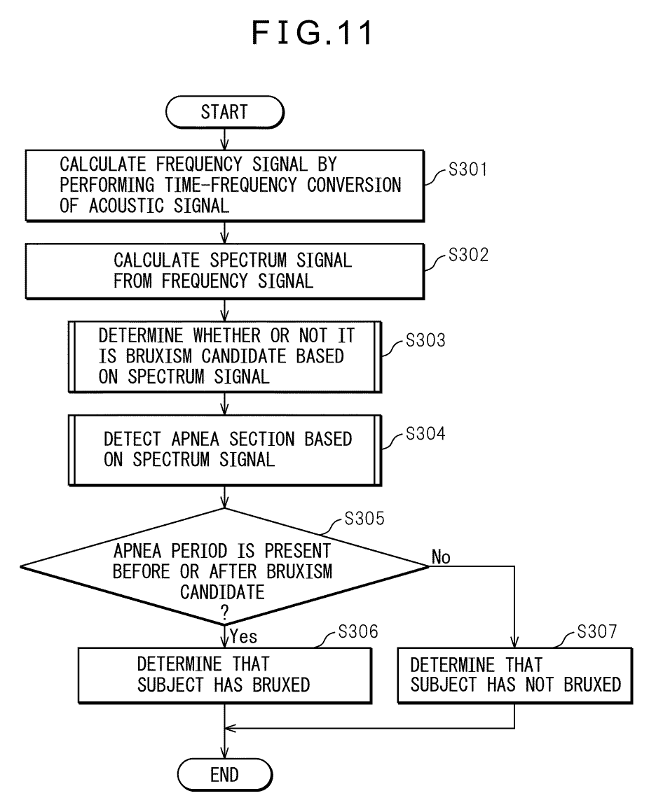

FIG. 11 is an operation flowchart of the bruxism detection process. The bruxism detection device 1 repeats executing this bruxism detection process during bruxism detection.

The time-frequency conversion unit 14 reads a sound signal that is collected by the microphone 11 and digitized by the analog/digital converter 12, from the buffer 13. Then, the time-frequency conversion unit 14 calculates a frequency signal by performing time-frequency conversion of the sound signal in frame units (step S301). The time-frequency conversion unit 14 outputs the frequency signal to the spectrum calculation unit 15.

The spectrum calculation unit 15 calculates spectrum signals, in frame units, from the frequency signal (step S302). Then, the spectrum calculation unit 16 outputs the spectrum signals to the bruxism candidate detection unit 16 and the breath detection unit 17.

The bruxism candidate detection unit 16 determines whether or not the signal period to include the present frame is a bruxism candidate, based on the spectrum signals (step S303). Then, the bruxism candidate detection unit 16 outputs a flag for indicating the result of determining whether or not the signal period to include the present frame is a bruxism candidate, and the number of the present frame, to the determining unit 18.

On the other hand, the breath detection unit 17 detects an apnea period, per breath detection period, based on the spectrum signals (step S304). Then, the breath detection unit 17 outputs the result of determining whether or not there is an apnea period, and the frame number to designate that breath detection period, to the determining unit 18, for each breath detection period.

The determining unit 18 determines whether or not there is an apnea period before or after a signal period that is determined to be a bruxism candidate (step S305). When there is an apnea period before or after a signal period that is determined to be a bruxism candidate (step S305—Yes), the determining unit 18 determines that the subject has bruxed (step S306). Then, the determining unit 18 outputs a bruxism detection signal to represent that determined result, to the output unit 19.

On the other hand, when no signal period is determined to be a bruxism candidate, or when no apnea period is present before or after a signal period that is determined to be a bruxism candidate (step S305—No), the determining unit 18 determines that the subject is not bruxing (step S307).

After step S306 or S307, the bruxism detection device 1 finishes the bruxism detection process.

As described above, this bruxism detection device detects a signal period to have a feature that is characteristic of bruxism, from the sounds collected by a microphone that is provided near the subject, as a bruxism candidate. Then, this bruxism detection device determines that the subject is bruxing, when a specific breathing state such as apnea is detected before or after the signal period to be a bruxism candidate. In this way, this bruxism detection device is able to determine whether or not the subject is bruxing based on sound alone, without imposing a physical load upon the subject.

Note that the present invention is not limited to the above embodiment. For example, the bruxism candidate determining unit of the bruxism candidate detection unit may detect a signal period to be a bruxism candidate, using only part of the whole-band signal power, the background noise power, the signal power of a specific frequency band, the attack sound detection result, the duration of attack sounds, the autocorrelation coefficient maximum value and the number of attacks.

For example, the bruxism candidate determining unit may use the following conditions as the conditions for determining whether a signal period including a frame of interest is a bruxism candidate.

(I) The whole-band signal power of the frame of interest is greater than the background noise power.

(II) The whole-band signal power of the frame of interest is greater than the background noise power, and the frame of interest is an attack sound.

(III) In addition to the conditions of above (I) or (II), the duration of attack sounds is equal to or longer than the threshold value Th2.

(IV) In addition to the conditions of above (I) or (II), the maximum value of the autocorrelation value acor(d) of the frame of interest is equal to or lower than the threshold value Th3.

In addition, the attack sound detection unit of the bruxism candidate detection unit may add the condition that the whole-band signal power of the frame of interest is greater than background noise power or the condition that the maximum value of the autocorrelation value acor(d) is equal to or lower than the above threshold value Th3, to the criteria for attack sound detection. In this case, when the duration of attack sounds is equal to or greater than the above threshold value Th2 and the number of attacks is equal to or longer than the threshold value Th4, the bruxism candidate determining unit may determine that a signal period corresponding to the attack sound duration T is a bruxism candidate.

Furthermore, the bruxism candidate determining unit may have a classifier that receives as input at least one of the whole-band signal power, the background noise power, the signal power of a specific frequency band, the duration of attack sounds, the autocorrelation coefficient maximum value, and the number of attacks, and determines whether or not the present frame is a bruxism candidate. The classifier may be a neural network such as a perceptron that has an input layer, a middle layer, and an output layer. In this case, a plurality of combinations of an input corresponding to the sound of bruxism and an output corresponding to a result of determining that a bruxism candidate is present, and a plurality of combinations of an input corresponding to a sound other than the sound of bruxism and an output corresponding to a result of determining that a bruxism candidate is not present, are prepared in advance, as supervised data. Then, the classifier may be learned in advance by back propagation using the supervised data. By this means, the classifier is able to output a determined result of high reliability, for any input. Note that the classifier to be provided in the bruxism candidate determining unit may be a support vector machine.

In addition, the storage unit may store in advance the spectrum signal of a certain period, which corresponds to various bruxism sounds, as a template. In this case, every time a spectrum signal of that certain period is acquired, the bruxism candidate detection unit performs pattern matching between the acquired spectrum signal and each template, and calculates the match level between the spectrum signal and the template. Then, when the maximum value of that match level is equal to or greater than a predetermined threshold value, the bruxism candidate detection unit may detect the frame in that certain period as a bruxism candidate frame. In this case, the certain period is set to, for example, 0.1 second to several seconds, corresponding to the period when bruxism lasts.

In addition, the breathing period detection unit may use the whole-band signal power, in addition to the autocorrelation coefficient, for breathing period detection. The sound when the subject is breathing is generally bigger than the sound when the subject is not breathing. Consequently, by detecting the magnitude of the whole-band signal power, the breathing period detection unit is able to detect breathing periods more accurately. In this case, the breathing period detection unit calculates the whole-band signal power of frames included in a period when the autocorrelation coefficient is equal to or greater than a breathing sound threshold value. Then, the breathing period detection unit detects a frame when the whole-band signal power exceeds a predetermined threshold value as a breathing period. Note that the predetermined threshold value is set to, for example, the average power of frames that correspond to the background noise.

Furthermore, the bruxism candidate determining unit may detect the signal periods to be a bruxism candidate in every predetermined bruxism candidate detection period. The bruxism candidate detection period is set to, for example, the same length as the breath detection period. Then, the bruxism candidate detection period and the breath detection period are set such that the frame in which the bruxism candidate detection period ends matches with the frame in which the breath detection period terminates.

In this case, the determining unit determines whether or not a bruxism candidate and an apnea period are detected, every time when the bruxism candidate detection period and the breath detection period terminate. Then, when both of a bruxism candidate and an apnea period are detected, the determining unit determines that the subject is bruxing.

In addition, the unit time in the signal power calculation, attack sound detection and autocorrelation value calculation may be different from a frame, which is the unit of time-frequency conversion. For example, the unit time in the signal power calculation, attack sound detection and autocorrelation value calculation may be double or triple the length of a frame. However, in this case, the unit time in the signal power calculation, attack sound detection and autocorrelation value calculation is set to be shorter than the breath detection period, the unit time for calculation of the number of attacks, and the analysis window for calculation of the duration of attack sounds.

Furthermore, according to another embodiment, the bruxism detection device may immediately determine, once a signal period to be a bruxism candidate is detected, that the subject is bruxing, without even using the result of determining the breathing state. In this case, the breath detection unit in the bruxism detection device illustrated in FIG. 2 may be omitted. However, in this case, the bruxism detection device determines that the subject is bruxing, preferably when all the conditions of steps S107 to S111 in the operation flowchart illustrated in FIG. 7 are fulfilled. Alternately, the bruxism detection device according to this embodiment determines that the subject is bruxing, preferably when, in addition to the condition of (II) or the condition of (II) of the above modification, the condition of (III) or the condition of (IV) is met.

Furthermore, a computer program to allow a computer to implement the functions of the time-frequency conversion unit, spectrum calculation unit, bruxism candidate detection unit. breath detection unit and determining unit provided in the bruxism detection device according to each embodiment may be provided recorded on a computer readable medium such as an optical recording medium, a magnetic recording medium and so on. However, a carrier wave is not included in this computer readable recording medium.

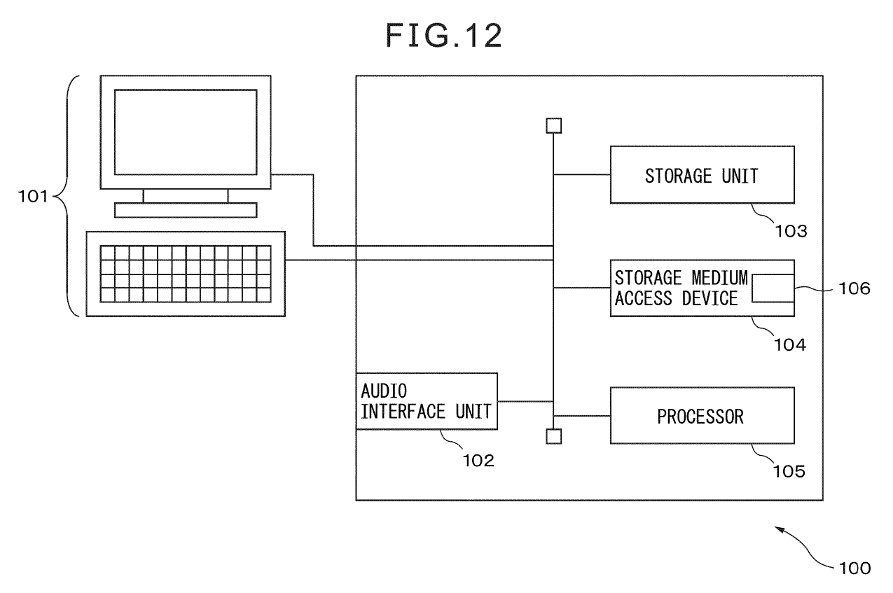

FIG. 12 is a configuration diagram of a computer that operates as a bruxism detection device as a computer program to implement the functions of the time-frequency conversion unit, spectrum calculation unit, bruxism candidate detection unit, breath detection unit and determining unit provided in the bruxism detection device according to each embodiment or its modification operates.

The computer 100 includes a user interface unit 101, an audio interface unit 102, a storage unit 103, a storage medium access device 104, and a processor 105. The processor 105 is connected with the user interface unit 101, audio interface unit 102, storage unit 103 and storage medium access device 104, for example, via a bus.

The user interface unit 101 has an input device such as a keyboard and a mouse, and a display device such as a liquid crystal display. The user interface unit 101 may have a device that integrates an input device and a display device, such as a touch panel display. Then, in response to a user operation to select an icon that is displayed on the display device and that commands execution of the bruxism detection process, the user interface unit 101 outputs an operation signal for starting the bruxism detection process, to the processor 105. The user interface unit 101 may display the result of determining whether or not bruxism is detected.

The audio interface unit 102 connects the computer 100 with a sound collection unit (not illustrated) such as a microphone, receives a sound signal to represent a sound that is produced from the subject from that sound collection unit, and passes the sound signal to the processor 105.

The storage unit 103 may, for example, have a readable and writable semiconductor memory, and a read-only semiconductor memory. Then, the storage unit 103 stores a computer program for performing the bruxism detection process, executed on the processor 105, sound signals to represent the sounds produced from the subject, and results of determining whether or not bruxism is detected.

The storage medium access device 104 is a device to access the storage medium 106, which is, for example, an optical disk, a semiconductor memory card or an optical storage medium. The storage medium access device 104, for example, reads the computer program for the bruxism detection process, stored in the storage medium 106 and executed on the processor 105, and passes that computer program to the processor 105.

The processor 105 implements the functions of the time-frequency conversion unit, spectrum calculation unit, bruxism candidate detection unit, breath detection unit and determining unit by executing a computer program for the bruxism detection process according to one of the embodiments above or its modification. Then, the processor 105 determines whether or not the subject has bruxed based on sound signals to represent the sounds produced from the subject. Then, the processor 105 stores that determined result in the storage unit 103 or has the user interface unit 102 display that determined result.

All examples and conditional language recited herein are intended for pedagogical purposes to aid the reader in understanding the invention and the concepts contributed by the inventor to furthering the art, and are to be construed as being without limitation to such specifically recited examples and conditions, nor does the organization of such examples in the specification relate to a showing of the superiority and inferiority of the invention. Although the embodiments of the present inventions have been described in detail, it should be understood that the various changes, substitutions, and alterations could be made hereto without departing from the spirit and scope of the invention.

What is claimed is:

1. A bruxism detection device comprising:
a sound collection unit that collects a sound produced from a subject and outputs a sound signal corresponding to collected sound;
a bruxism candidate detection unit that detects a period of a sound having a feature that is characteristic of bruxism, from the sound signal, as a bruxism candidate period;
a breath detection unit that detects a period of a sound having a feature corresponding to a predetermined breathing state, from the sound signal, as a specific breathing period; and
a determining unit that determines that the subject has bruxed, when the specific breathing period is present before or after the bruxism candidate period.

2. The bruxism detection device as claimed in claim 1, wherein the bruxism candidate detection unit comprises:
a feature extraction unit that determines, as a feature amount, at least one of a signal power of each frequency band in a first period of the sound signal, which is divided into predetermined units, a signal power of a whole frequency band and a background noise signal power in the first period, a duration of an attack sound that continues up to the first period, a number of times when the attack sound occurs, and a maximum value of an autocorrelation coefficient between the sound signal of the first period and the sound signal of a period that is earlier than the first period; and
a bruxism candidate determining unit that, when the feature amount fulfills a predetermined condition, determines that a period of the sound signal including the first period is the bruxism candidate period.

3. The bruxism detection device as claimed in claim 2, wherein,
the feature extraction unit extracts the signal power of the whole frequency band and the background noise signal power for the first period as the feature amount, and
the bruxism candidate determining unit determines that the feature amount fulfills the predetermined condition, when the signal power of the whole frequency band of the first period is equal to or greater than the background noise signal power.

4. The bruxism detection device as claimed in claim 2, wherein,
the feature extraction unit extracts the signal power of each frequency band, the signal power of the whole frequency band and the background noise power in the first period as the feature amount, and
the bruxism candidate determining unit determines that the feature fulfills the predetermined condition, when the signal power of the whole frequency band of the first period is equal to or greater than the background noise signal power, and, among each frequency band in the first period, the number of frequency bands of which the signal power is greater than the signal power of corresponding frequency band in a second period, which is earlier than the first period, is equal to or greater than a predetermined number.

5. The bruxism detection device as claimed in claim 2, wherein,
the feature extraction unit extracts the signal power of each frequency band, the signal power of the whole frequency band, and the background noise power in the first period, and the duration, as the feature amount, and
the bruxism candidate determining unit determines that the feature amount fulfills the predetermined condition, when the signal power of the whole frequency band of the first period is equal to or greater than the background noise signal power, among each frequency band in the first period, the number of frequency bands of which the signal power is greater than the signal power of corresponding frequency band in a second period, which is earlier than the first period, is equal to or greater than a predetermined number, and the duration is equal to or longer than a duration of bruxism.

6. The bruxism detection device as claimed in claim 2, wherein,
the feature extraction unit extracts the signal power of each frequency band, the signal power of the whole frequency band, and the background noise power in the first period, the duration, and the maximum value of the autocorrelation coefficient as the feature amount, and
the bruxism candidate determining unit determines that the feature amount fulfills the predetermined condition, when the signal power of the whole frequency band of the first period is equal to or greater than the background noise signal power, among each frequency band in the first period, the number of frequency bands of which the signal power is greater than the signal power of corresponding frequency band in a second period, which is earlier than the first period, is equal to or greater than a predetermined number, the duration is equal to or longer than the duration of bruxism, and the maximum value of the autocorrelation coefficient is equal to or lower than a predetermined value.

7. The bruxism detection device as claimed in claim 2, wherein,
the feature extraction unit extracts the duration and the number of attacks as the feature amount, and
the bruxism candidate determining unit determines that the feature amount fulfills the predetermined condition, when the duration is equal to or longer than a duration of bruxism, and the number of attacks is equal to or greater than a predetermined number, the predetermined number being equal to or greater than 2.

8. The bruxism detection device as claimed in claim 2, wherein the bruxism candidate determining unit comprises a classifier that, by receiving the feature amount as input, outputs a result of determining whether or not the period of the sound signal including the first period is the bruxism candidate period.

9. The bruxism detection device as claimed in claim 1, wherein the breath detection unit detects a state in which the subject is not breathing as the predetermined breathing state and a period of a sound corresponding to the state in which the subject is not breathing, as the specific breathing period.

10. The bruxism detection device as claimed in claim 9, wherein, with respect to a second period of the sound signal which is divided in the predetermined units, when the sound signal of the second period and the sound signal of a third period before or after the second period have periodicity, the breath detection unit detects the second period and the third period as the breathing period in which the subject is breathing, and, when a difference in time between two neighboring breathing periods is equal to or greater than a predetermined length of time, detects an interval of the two breathing periods as the specific breathing period.

11. A bruxism detection method comprising:
collecting a sound produced from a subject, and, from a sound signal corresponding to collected sound detecting a period of a sound having a feature that is characteristic of bruxism, as a bruxism candidate period;
detecting a period of a sound having a feature corresponding to a predetermined breathing state, from the sound signal, as a specific breathing period; and
determining that the subject has bruxed, when the specific breathing period is present before or after the bruxism candidate period.

12. The bruxism detection method as claimed in claim 11, wherein the detecting the specific breathing period comprises:
determining, as a feature amount, at least one of a signal power of each frequency band in a first period of the sound signal, which is divided into predetermined units, a signal power of a whole frequency band and a background noise signal power in the first period, a duration of an attack sound that continues up to the first period, a number of times when the attack sound occurs, and a maximum value of an autocorrelation coefficient between the sound signal of the first period and the sound signal of a period that is earlier than the first period; and
determining a period of the sound signal including the first period as the bruxism candidate period when the feature amount fulfills a predetermined condition.

13. The bruxism detection method as claimed in claim 12, wherein,
the determining the feature amount extracts the signal power of the whole frequency band and the background noise signal power for the first period as the feature amount, and
the determining the bruxism candidate period determines that the feature amount fulfills the predetermined condition, when the signal power of the whole frequency band of the first period is equal to or greater than the background noise signal power.

14. The bruxism detection method as claimed in claim 12, wherein,
the determining the feature amount extracts the signal power of each frequency band, the signal power of the whole frequency band and the background noise power in the first period as the feature amount, and
the determining the bruxism candidate period determines that the feature fulfills the predetermined condition, when the signal power of the whole frequency band of the first period is equal to or greater than the background noise signal power, and, among each frequency band in the first period, the number of frequency bands of which the signal power is greater than the signal power of corresponding frequency band in a second period, which is earlier than the first period, is equal to or greater than a predetermined number.

15. The bruxism detection method as claimed in claim 12, wherein,
the determining the feature amount extracts the signal power of each frequency band, the signal power of the whole frequency band, and the background noise power in the first period, and the duration, as the feature amount, and
the determining the bruxism candidate period determines that the feature amount fulfills the predetermined condition, when the signal power of the whole frequency band of the first period is equal to or greater than the background noise signal power, among each frequency band in the first period, the number of frequency bands of which the signal power is greater than the signal power of corresponding frequency band in a second period, which is earlier than the first period, is equal to or greater than a predetermined number, and the duration is equal to or longer than a duration of bruxism.

16. The bruxism detection method as claimed in claim 12, wherein,
the determining the feature amount extracts the signal power of each frequency band, the signal power of the whole frequency band, and the background noise power in the first period, the duration, and the maximum value of the autocorrelation coefficient as the feature amount, and
the determining the bruxism candidate period determines that the feature amount fulfills the predetermined condition, when the signal power of the whole frequency band of the first period is equal to or greater than the background noise signal power, among each frequency band in the first period, the number of frequency bands of which the signal power is greater than the signal power of corresponding frequency band in a second period, which is earlier than the first period, is equal to or greater than a predetermined number, the duration is equal to or longer than the duration of bruxism, and the maximum value of the autocorrelation coefficient is equal to or lower than a predetermined value.

17. The bruxism detection method as claimed in claim 12, wherein,
the determining the feature amount extracts the duration and the number of attacks as the feature amount, and
the determining the bruxism candidate period determines that the feature amount fulfills the predetermined condition, when the duration is equal to or longer than a duration of bruxism, and the number of attacks is equal to or greater than a predetermined number, the predetermined number being equal to or greater than 2.

18. The bruxism detection method as claimed in claim 11, wherein the detecting the specific breathing period detects a state in which the subject is not breathing as the predetermined breathing state and a period of a sound corresponding to the state in which the subject is not breathing, as the specific breathing period.

19. The bruxism detection method as claimed in claim 18, wherein, with respect to a second period of the sound signal which is divided in the predetermined units, when the sound signal of the second period and the sound signal of a third period before or after the second period have periodicity, the detecting the specific breathing period detects the second period and the third period as the breathing period in which the subject is breathing, and, when a difference in time between two neighboring breathing periods is equal to or greater than a predetermined length of time, detects an interval of the two breathing periods as the specific breathing period.

20. A computer readable recording medium that is stored with a computer program for a bruxism detection process, the computer program causing a computer to execute:
collecting a sound produced from a subject, and, from a sound signal corresponding to collected sound detecting a period of a sound having a feature that is characteristic of bruxism, as a bruxism candidate period;
detecting a period of a sound having a feature corresponding to a predetermined breathing state, from the sound signal, as a specific breathing period; and
determining that the subject has bruxed, when the specific breathing period is present before or after the bruxism candidate period.

21. A bruxism detection device comprising:
a sound collection unit that collects a sound produced from a subject and outputs a sound signal corresponding to collected sound;
a feature extraction unit that determines, as a feature amount, at least one of a signal power of each frequency band in a first period of the sound signal, which is divided into predetermined units, a signal power of a whole frequency band and a background noise signal power in the first period, a duration of an attack sound that continues up to the first period, a number of times when the attack sound occurs, and a maximum value of an autocorrelation coefficient between the sound signal of the first period and the sound signal of a period that is earlier than the first period; and
a determining unit that, when the feature amount fulfills a predetermined condition, determines that the subject is bruxing.

22. A bruxism detection device comprising:
a processor adapted to:
detect a period of a sound having a feature that is characteristic of bruxism, as a bruxism candidate period, from a sound signal that is generated by collecting a sound produced from a subject;
detect a period of a sound having a feature corresponding to a predetermined breathing state, from the sound signal, as a specific breathing period; and
determine that the subject has bruxed, when the specific breathing period is present before or after the bruxism candidate period.
